(19) United States
(12) Patent Application Publication
(10) Pub. No.: US 2008/0249578 A1
(43) Pub. Date: Oct. 9, 2008

(54) Fastener for securing two separate workpieces

(76) Inventor: Madhavan Pisharodi, Brownsville, TX (US)

Correspondence Address: JACKSON WALKER, L.L.P., 112 E. PECAN, SUITE 2400, SAN ANTONIO, TX, 78205, US

(21) Appl. No.: 12/157,229

(22) Filed: Jun. 9, 2008

Related U.S. Application Data
(63) Continuation of application No. 10/729,038, filed on Dec. 5, 2003.
Application No. 10/729,038 is a continuation-in-part of application No. 10/419,413, filed on Apr. 21, 2003, now Pat. No. 6,989,013.

(51) Int. Cl. A61B 17/58

(52) U.S. Cl. 606/310; 606/301

(57) ABSTRACT

A locking or fastener system for joining or attaching a first workpiece spaced apart from a second workpiece. A threaded opening is provided in the first workpiece for receiving a screw/collar combination. The screw has a threaded end and a head end. The screw head is rotatingly attached within a cavity within the collar. External threads on the wall of the collar cooperate with the threads in the opening in the first workpiece. When the collar is secured to the first workpiece, the screw may be rotated to engage the second workpiece. By using a wrench having an outer body with extending blades and an inner rod having a screw face, the collar and screw may be tightened to join two spaced-apart workpieces.

This is a continuation application claiming priority to and incorporating by reference herein co-pending U.S. continuation-in-part patent application Ser. No. 10/729,038, filed Dec 5, 2003, and U.S. patent application Ser. No. 10/419,413, filed Apr. 21, 2003.

### BACKGROUND OF THE INVENTION

The present invention relates to a fastener system for joining two separate, spaced-apart workpieces. More particularly, the present invention relates to a screw and collar configuration which attaches to a first workpiece by rotation of a threaded collar section in a first rotational direction thereby securing the screw/collar assembly to the first workpiece while rotation of the screw member in a second rotational direction while urging it against a second workpiece secures the screw to a second workpiece and resultantly joins the first workpiece to the second workpiece.

Initial development of the present invention was done with a medical appliance for bridging and stabilizing spaced-apart bone segments. A locking system was developed to provide stabilization of spaced-apart bone segments while still allowing some flexion and rearward extension of the bones with some lateral displacement. Subsequently, it was discovered that the locking system would be effective in securing any two spaced-apart workpieces, not just bone segments, including but not limited to woods, metals, plastics, and any other composition that will take or hold a thread.

### SUMMARY OF THE INVENTION

Accordingly, the present invention provides a fastener or a screw locking system for joining and securing two, separate, spaced-apart workpieces. A screw having a head portion and a threaded body section has a slot in the head end to facilitate rotation of the screw as is conventionally known. A locking collar member is provided with an inner cavity for receiving and retaining the screw head. The outer wall of the collar is threaded to cooperate with corresponding threads in the first workpiece to releasably secure the collar to the first workpiece by rotation of the collar in a first rotational direction. The collar further has a slot along the top surface of the collar to facilitate the rotation of the collar. There is sufficient clearance between the outer surface of the screw head and the inner surface of the collar cavity to allow the screw to rotate within the collar cavity, but not so great a clearance as to allow the head to fall out or be pushed out of the collar cavity. The screw head/collar assembly may be threaded into the first workpiece as the threaded body section of the screw is threaded into the second workpiece. The head screw is rotatably secured within the collar and by tightening the collar into the first workpiece the screw is secured to the first workpiece. As the screw head is rotated in a second rotational direction (which may be the same as the first rotational direction) and the thread body section is urged against the second workpiece, the two workpieces are joined together. This happens because there is sufficient clearance for the screw head to rotate within the collar cavity without causing the collar to loosen from the first workpiece.

The use of complimental screw head shapes (concave or convex) with corresponding collar cavity shapes (convex or concave) ensures that the screw head is securely retained within the collar cavity. Forces urging the screw to loosen are not translated to the collar, therefore, the two workpieces stay joined in the most adverse conditions.

A unique wrench or tool is provided to allow for the easy rotation of the collar in a first direction while allowing the screw head to be rotated in a second direction. Normally, the present invention will use screws and collars of the opposite thread hand, but there may be some applications where the screw and collar are the same hand thread. The number of threads on the collar section may be different than the number of threads on the screw to reduce the possibilities of the parts loosening.

### BRIEF DESCRIPTION OF THE DRAWINGS

FIG. 1 is a partial cutaway, perspective view of one embodiment of the present invention.

FIG. 2A illustrates a cross sectional side elevation view of the embodiment of FIG. 1 with the collar secured in the first workpiece and the screw starting into the second workpiece.

FIG. 2B depicts the two workpieces joined together by the fastener of the present invention.

FIG. 3 shows a partial cutaway, perspective view of another embodiment of the present invention.

FIG. 4 illustrates a detailed partial cross sectional side elevation view of the embodiment of FIG. 3.

FIG. 5 is a side elevation illustration of the screw member of the embodiment of FIG. 3.

FIG. 6 shows a side elevation plan view of the wrench of the present invention.

FIG. 7 is a bottom plan view of the wrench of FIG. 6 showing opposite directions of rotation for the central rod and the outer barrel of the wrench.

### DETAILED DESCRIPTION OF THE PREFERRED EMBODIMENT

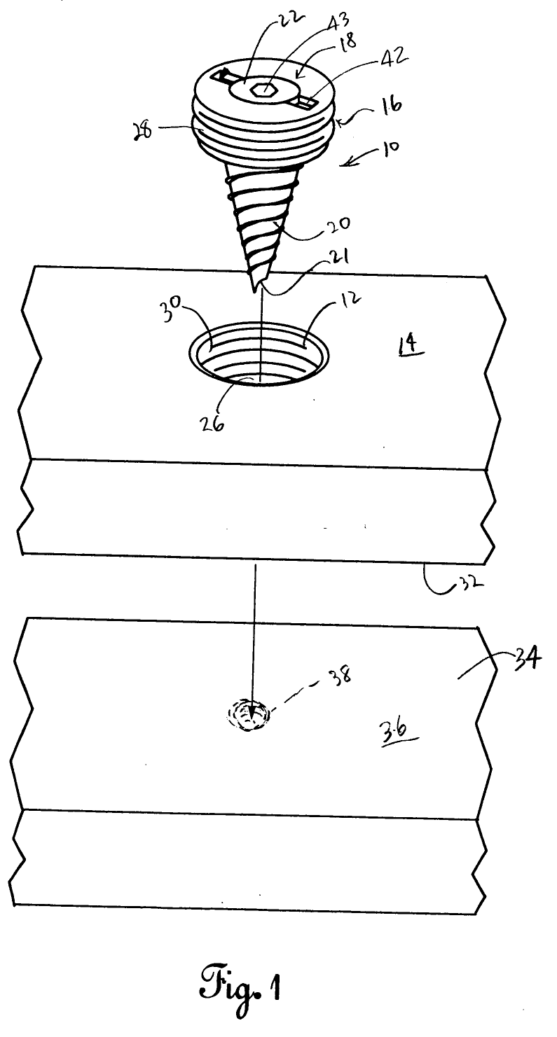

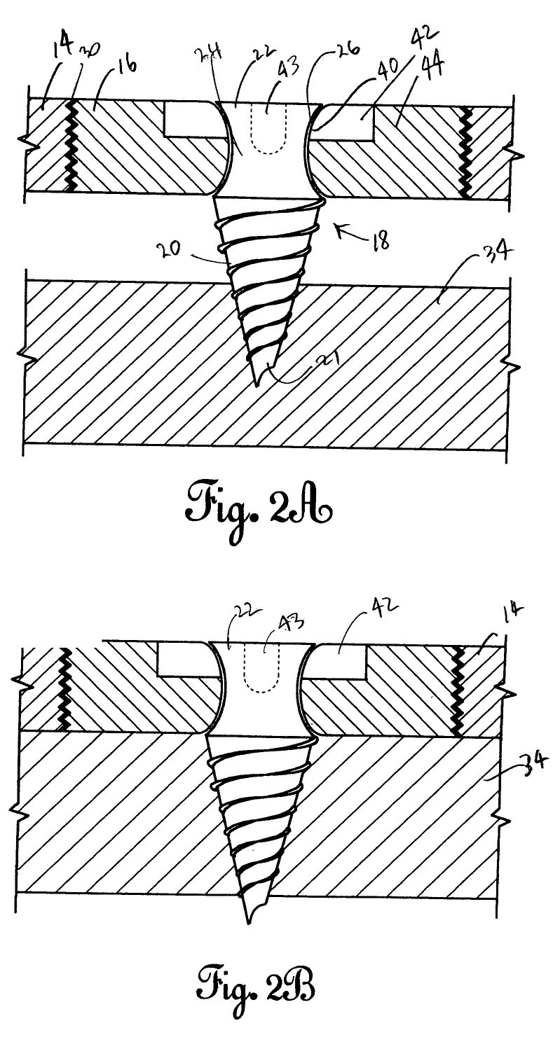

The present invention, described herein, is used in conjunction with workpieces of whatever type which may be joined together by a threaded fastener. FIG. 1 illustrates the unique screw and collar fastener 10 of the present invention in an exploded perspective view. The fastener 10 is first secured in a threaded opening 12 in a first workpiece 14. The fastener is secured in the first workpiece by the collar section 16. A separate screw 18 is rotatably secured within collar section 16. The screw 18 has a threaded first end 20, an opposite head end 22, and a head portion 24 (FIG. 2A) which is rotatably secured within an opening 26 in the threaded collar section 16. As may be seen in the drawings, the collar section 16 has external threads 28 which engage the complimentary internal locking threads 30 of the opening 12.

In FIG. 1, it may be seen that the thread end 20 of the screw extends beyond the underside 32 of the workpiece 14 as it passes through opening 12. A second workpiece 34 may be joined to the first workpiece 14 as the pointed end 21 of the screw 18 is urged to penetrate the upper surface 36 of workpiece 34. It should be understood that in some applications the second workpiece may be provided with a predrilled reception orifice shown as broken lines 38 in FIG. 1. In such situations the whole range of screw types may be used in the present inventive system.

Turning to FIGS. 2A and 2B, the operation of the present inventive system may be seen. In FIG. 2A, screw 18 having a concave head portion 24, is retained in a cooperating, complimentary convex opening 26 in the thread collar section 16. The collar section has a rounded shoulder 40 to form the wall of the opening 26. A rotation slot 42 is cut into the body 44 so that the collar 16 may be rotated and secured in the threaded opening. (Again, one of ordinary skill in the art will recognize that opening 30 may not need to be countersunk to allow the present inventive system to operate.) As the collar section 16 rotates and tightens into the first workpiece 14, the screw 18 is free to rotate about its head section 24 within the slightly larger complimentary opening 26 in the collar section 16.

Once the collar/screw combination is secured in the first workpiece, the screw 18 may be rotated by placing an appropriate tool into the rotation slot 42 in head end 22. By urging upon the screw head end 22, the tip 21 is screwed or fastened into the second workpiece 34. FIG. 2B shows the two pieces 14 and 34 joined together. In some applications it may be appropriate to gradually tighten a portion of the collar 16 into the first workpiece 14 while gradually tightening the screw 18 into the second workpiece 34.

Further, the use of opposite sets of threads may be employed such that the possible slight rotation of the screw to loosen may be translated to the collar to tighten the collar in workpiece 14.

FIG. 3 illustrates an alternative embodiment 50 of the present invention which uses a substantially spherical or convex shaped screw head portion 24A retained in a complimentary concave shaped opening 26A in the collar section 16A. As discussed above, the two spaced-apart workpieces 14 and 34 may be joined by securing the collar 16A in workpiece 14 and tightening the screw into workpiece 34 and drawing the pieces together. FIG. 3 illustrates the concept of the collar 16A having a first rotational direction A for tightening and the screw 24A having a second rotational direction B for tightening.

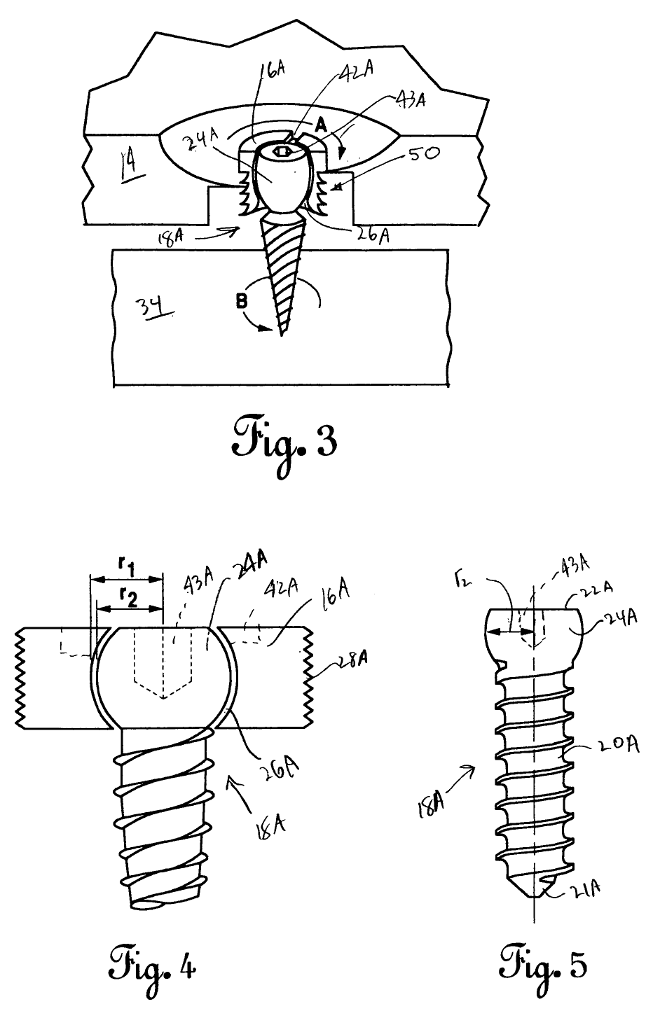

FIG. 4 shows a detailed, partial, cross-sectional view of the spherical headed screw 24A of FIG. 3. As may be seen in FIG. 4, the opening 26A has a radius r₁ which is slightly larger than the radius r₂ of the spherical head 24A. This creates a slight gap. This allows the screw to rotate freely within the collar section. FIG. 5 is simply a side elevation view of the screw 18A showing the head end 22A, the tightening slot 43A, the head portion 24A, the threaded end 20A, and the screw pointed end 21A. The head portion 24A is provided with a convex, generally spherical radius r₁. As shown in FIG. 4, radius r₁ cooperates with a corresponding, complimentary, inner concave radius of the opening 26A to limit axial and flexion movement of the screw collar 16A while the collar remains engaged in the opening 26A in the first workpiece 14.

To facilitate the insertion of the insertion locking and fastener systems of the embodiments described above, a unique wrench has been developed.

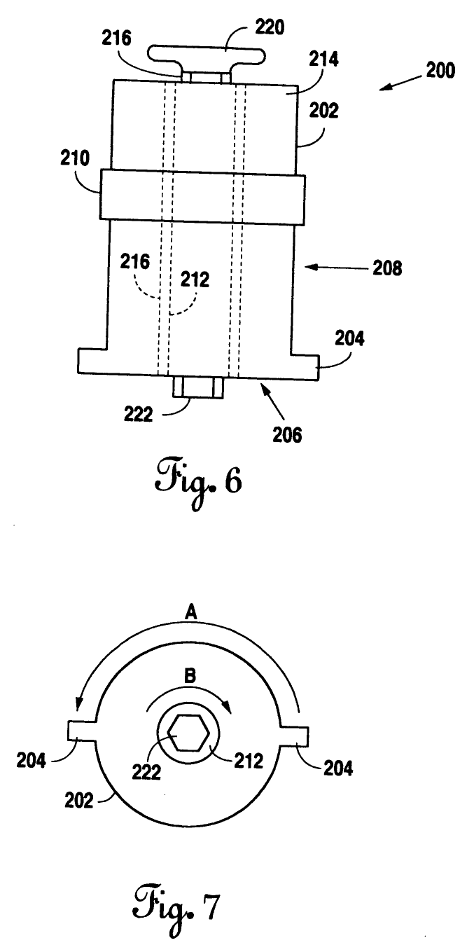

In FIG. 6, wrench 200 has a generally cylindrical outer barrel housing 202 with outwardly extending blades 204 at the base 206 of the barrel. Around a central portion 208 is a grip member which is used to rotate the outer barrel 202 and the blades 204.

The barrel 202 has an inner orifice 212 extending from a top end 214 through the base 206. An elongated, generally cylindrical rod 216 extends through the orifice 212 with a handle member 220 on a top end and a screw face 222 on the opposite, bottom end. The screw face extends out of the orifice 212 and beyond the base 206. In one embodiment of the wrench, the rod and screw face are similar to a traditional Allen wrench with a number of flat surfaces which engage the flat surfaces of a cooperating Allen head screw. It should be understood that the rod and screw face may be other cooperating configurations including a socket wrench-like configuration well known in the fastener art.

FIG. 7 illustrates a bottom end view of the wrench 200 showing the arrangement of the screw face 222 and blades 204. It should be readily understandable that when the screw 18 or 18A of the present locking system has an Allen-type recess 43 or 43A in the screw head 24A, the screw face 222 has a complimentary Allen-type projection for rotation of the screw 18 or 18A. However, in order to tighten the collar portion 16 or 16A within the opening 12 in the workpiece 14, the blades 224 engage the slot 40 in the top face of the collar 42A.

Thus, by the gradual tightening of the screw 18 or 18A separately from the tightening of the collar 16 or 16A, the locking system is utilized to engage the screw with the second workpiece 34. It has been found that when the threads of the screw 20 or 20A and the threads of the collar 28 or 28A are opposite hand, the likelihood of the screw loosening from the second workpiece is considerably reduced. (See directional arrows A and B in FIG. 3.)

As previously stated, while the threads 20 or 20A of the screw 18 or 18A and the collar threads 28 and 28A may be the same hand, the threads per millimeter of the screw are different than the threads per millimeter of the collar. This will achieve a degree of improved retention of the screw in the second workpiece while allowing the desired movement of the screw within the concave gap between the screw head and the inner walls of the collar cavity.

Although the invention has been described with reference to a specific embodiment, this description is not meant to be construed in a limiting sense. On the contrary, various modifications of the disclosed embodiments will become apparent to those skilled in the art upon reference to the description of the invention. It is therefore contemplated that the appended claims will cover such modifications, alternatives, and equivalents that fall within the true spirit and scope of the invention.

1. A locking system for releasably attaching a first workpiece to a second workpiece comprising:
an opening in said first workpiece for receiving a unitary screw-collar ring assembly comprising:
a screw member having a threaded first end threaded in a first direction and an opposite head end, and
a collar ring member having an inner ring aperture and external threads, said collar ring member-rotatingly coupled to and affixed about said opposite head of said screw member to retain said opposite head in said inner ring aperture of said collar ring member and hold said screw member and said collar ring member as a unitary assembly; and
complimentary locking threads in said opening in said first workpiece for engaging said collar ring member to said first workpiece at said head such that axial and rotational movement of said screw member is restricted when said first workpiece is affixed to said second workpiece by urging and rotating said threaded first end of said screw member into said second workpiece, said complimentary locking threads threaded in a second direction opposite said first threaded direction of said screw member.

2. The locking system of claim 1, wherein said opposite head end of said screw member is substantially spherical.

3. The locking system of claim 2 wherein said threaded collar ring member has a concave screw head retaining cavity, said substantially spherical opposite head end of said screw adapted to be rotatingly secured within said cavity.

4. The system of claim 1 further comprising a wrench having an outer body with outwardly extending blades and an inner rod rotatably extending through an inner passage of said body, said rod having a screw face portion extending beyond a base of said body and adapted to engage said opposite head end of said screw member, said blades adapted to engage a slot in a top face of said collar ring.

5. A fastener system for releasably joining a first workpiece to a second workpiece comprising:
a unitary screw-collar ring assembly comprising:
a screw member having a head and a threaded body section, said head having a top surface, an underlying shoulder, and screw-tool receiving cavity to facilitate rotation of said screw; and
a locking collar ring coupled to and rotatingly affixed about said top surface and said underlying shoulder of said head of said screw, said locking collar ring having a top surface, a top opening, a collar-tool receiving slot, a bottom opening, a smooth, inner cavity, and a threaded outer wall, said threaded outer wall cooperating with a complimentary threaded inner surface of a screw-collar ring assembly receiving opening in said first workpiece to releasably secure said screw-collar ring assembly in said first workpiece when said locking collar ring is rotated in said screw-collar ring assembly receiving opening in a first direction of rotation, said inner cavity adapted to surround and rotatingly retain said head of said screw in said inner cavity of said locking ring collar and hold said screw member and said locking ring as a unitary assembly with said screw-tool receiving cavity exposed through said top opening of said locking collar ring and said threaded body section of said screw member extending outwardly from said bottom opening of said locking collar ring sufficiently to engage and join said second workpiece when said threaded body section of said screw member is rotatably urged against said second workpiece in a second direction of rotation, opposite said first direction of rotation of said screw-collar ring assembly in said screw-collar ring assembly receiving opening.

6. A fastener system of claim 5 further comprising a wrench having an outer body with outwardly extending blades, an inner rod rotatably extending through an inner passage of said body, said rod having a face portion extending beyond a base of said body and adapted to engage said tool receiving cavity of said head of said screw member, said blades adapted to engage a slot in said top surface of said locking collar ring.

7. The locking system of claim 5 wherein said opposite head end of said screw member has a circumferential concave groove.

8. The locking system of claim 5 wherein said threaded collar ring member has a convex screw head retaining cavity, said circumferential concave groove adapted to be rotatingly secured within said convex screw head retaining cavity.
